(19) United States
(12) Patent Application Publication
(10) Pub. No.: US 2025/0127746 A1
(43) Pub. Date: Apr. 24, 2025

(54) TOPICAL CONTACT SENSITIZERS TO TREAT CANCER

(71) Applicant: Squarex Pharmaceutical Corporation, Pine Springs, MN (US)

(72) Inventor: Hugh McTavish, Pine Springs, MN (US)

(21) Appl. No.: 18/918,564

(22) Filed: Oct. 17, 2024

Related U.S. Application Data
(60) Provisional application No. 63/647,613, filed on May 14, 2024.
(60) Provisional application No. 63/591,319, filed on Oct. 18, 2023.

(51) Int. Cl. A61K 31/21; A61K 9/00; A61K 31/04; A61K 31/122; A61P 37/04

(52) U.S. Cl. CPC A61K 31/21; A61K 9/0014; A61K 31/04; A61K 31/122; A61P 37/04

(57) ABSTRACT

Methods of treating cancers or precancerous conditions and treating cancers in partial or complete remission are disclosed. The methods involve topical application of topical contact sensitizers, such as squaric acid dibutylester.

Too many people die from cancer, treatments are not effective enough; the side effects are terrible, especially from chemotherapy, including severe nausea and immune suppression, and often death as a side effect of the treatment; and recurrences after a patient is declared cancer-free or the cancer in remission are too frequent. And treatments are too expensive.

There is a need for treatments that work with the body's natural immune system instead of weakening it, that have low side effects, that reduce recurrence rates, and that are inexpensive and easy for patients to administer themselves.

### SUMMARY

The invention is to apply squaric acid dibutylester (SADBE) or other topical immunosensitizers to the skin of a patient to treat cancers or precancerous lesions or prevent recurrence of cancers. The method is especially effective with cancers caused by human papillomavirus (HPV), which include cervical cancer, as well as vulvar and penile cancers and a subset of head and neck cancers. But it can be effective with all cancers because we know that the immune system can control cancers and kill malignant cells, as shown by the success of checkpoint inhibitors and chimeric antigen receptor T-cell therapy (CAR-T) for treating cancers. SADBE and other topical immunosensitizers broadly strengthen the immune system in ways known to suppress cancers.

Data is shown herein that intradermal injection of Candida antigen to skin of the arm causes regression of pre-cancerous cervical lesions. Importantly, Candida antigen alone was more effective than Candida antigen mixed with targeted peptides of HPV. This shows that it is the non-specific effect of Candida antigen in altering and improving immunity that is the mechanism of action. The effect was linked with T-cell expression of interferon-gamma, as shown in the data of Example 1 below.

Intradermal injection and intralesional injection of Candida antigen has previously been shown to be effective to treat common warts, which are caused by HPV. (U.S. Pat. No. 6,350,451.) It acts by causing a delayed-type hypersensitivity response—also known as a type IV immune response. Topical application of SADBE and other topical immunosensitizers have also been shown to be effective to treat common warts and also act by causing a delayed-type hypersensitivity response. (Buckley, D. A. et al. 2001, The therapeutic use of topical contact sensitizers in benign dermatoses. Br. J. Dermatology 145:385-405. Lee et al. Journal of the American Academy of Dermatology, 1999, Vol. 41, pages 595-599.) Topical application of SADBE to the arm has also been shown to prevent and reduce the number of oral herpes outbreaks, cold sores. (U.S. Pat. No. 10,245,314; Chang A L S, Honari G, Guan L, Zhao L, Palli M A, Horn T D, Dudek A Z, McTavish H. A phase 2, multi-center, placebo-controlled study of single dose squaric acid dibutyl ester (SADBE) to reduce frequency of outbreaks in subjects with recurrent herpes labialis. Journal of the American Academy of Dermatology 2020 December;83 (6): 1807-1809.) Accordingly, topical application of SADBE and other topical immunosensitizers is also effective, like intradermal injection of Candida antigen, to cause regression of pre-cancerous lesions caused by HPV and to treat cancer.

Topical application of contact immunosensitizers is also effective to treat cancers not caused by HPV.

There is nothing about the mechanism that is specific to a particular tumor antigen or target, including HPV antigens. It just shifts the immune response from a type 2 or antibody response to a type 1 or cellular response and improves the ability of the immune system to mount a cellular response and express interferon-gamma when immune cells encounter antigens, whether HPV antigens, herpes antigens, fungal antigens, or cancer antigens (i.e., mutated proteins of malignant cells).

Of course it is not a cure as a sole treatment for late-stage aggressive cancers. But it can improve cancer outcomes, and thus is effective to treat cancer. It is especially effective in patients with reasonably healthy immune systems and a relatively low tumor burden. Thus, it prevents recurrence (reduces recurrence rates) of cancers in patients who have been treated for cancer and are considered cancer-free or in partial or complete remission, both for cancers caused by HPV and those that are not, and to treat cancers, both those caused by HPV and those that are not. It is also effective against slow-growing tumors such as those of prostate cancer and low-grade non-Hodgkins lymphoma.

One embodiment of the invention provides a method of treating a mammal for a cancer comprising: applying a topical immunosensitizer to skin of the mammal.

One embodiment of the invention involves a method of treating a mammal for a cancer in remission comprising: applying a topical immunosensitizer to skin of the mammal.

One embodiment of the invention provides a method of treating a cancer or precancerous lesions in a mammal comprising: applying a topical immunosensitizer to skin of the mammal; wherein the mammal is afflicted with a cancer caused by human papilloma virus (HPV) or precancerous lesions caused by HPV or was afflicted with said cancer and the cancer is now in remission or was afflicted with said precancerous lesions and the lesions have regressed or been removed.

One embodiment of the invention provides a method of treating a mammal for a cancer in remission comprising: applying a topical immunosensitizer to skin of the mammal; wherein the mammal has been diagnosed with a cancer, treated for the cancer, and the cancer is in remission (partial remission or complete remission) at the time of the applying step.

One embodiment of the invention provides a method of treating a mammal for cancer, the method comprising: (a) applying a topical immunosensitizer to skin of the mammal; and (b) administering to the mammal a checkpoint inhibitor; wherein the mammal has been diagnosed with a cancer and is in recognized need for treatment of the cancer.

### BRIEF DESCRIPTION OF THE DRAWINGS

FIG. 1. CONSORT diagram.

FIG. 2A. Histological responses and subgroup comparisons. FIG. 2A shows the analyses The stringent criterion was used in which only the complete responders (regression to no CIN) were considered to have had responses.

FIG. 2B. Histological responses and subgroup comparisons. FIG. 2B shows the analyses of the per-protocol analyses. The stringent criterion was used, in which only the complete responders (regression to no CIN) were considered to have had responses.

FIG. 3. Analyses of HPV clearance by risk groups and HPV 16 viral load. This figure shows the percentages of HPV clearance (i.e., detected at entry but not at the exit visit) by HPV types grouped as (1) HPV 16, (2) HPV 16-related types other than HPV 16, (3) high-risk HPV types not including HPV 16/HPV16-related, and (4) low-risk HPV types.

FIG. 4. Analyses of HPV clearance by risk groups and HPV 16 viral load. This figure shows HPV 16 viral loads in patients who were HPV 16-positive at entry.

### DETAILED DESCRIPTION

In Example 1 below, it is shown that intradermal injection of Candida antigen causes partial and complete regression of cervical intraepithelial neoplasia (CIN) 2 and 3 lesions (CIN2 and CIN3 lesions). With Candida extract complete regression occurred in 18 of 29 subjects, which compares to 34 of 117 in historical placebo, (P=0.001). PepCan, which is a mixture of Candida extract and four human papillomavirus type 16 E6 peptides, was less effective than Candida extract alone, with complete regression in 11 of 24 patients (P=0.24 lesser efficacy versus Candida alone and P=0.11 greater efficacy versus historical placebo). So adding HPV peptides (selected because an immune response against those peptide sequences had previously been shown to correlate with CIN regression) to Candida decreased the efficacy of Candida extract in causing regression of the pre-cancerous lesions.

In contrast, it is shown that adding the peptides to Candida extract does cause increased immune response to those peptides. By ELISPOT assay for secretion of interferon gamma, 7 of 33 patients receiving PepCan had a response against an HPV E16 peptide before vaccination and that went to 18 of 33 after. That means 11 of 26 patients who did not previously have a detectable immune response against the HPV E16 protein developed immune recognition of that protein after PepCan vaccination.

For vaccination with Candida antigen alone, 13 of 38 had an ELISPOT recognition of HPV E16 before vaccination and 17 of 38 had one after vaccination. So with Candida extract alone, only 4 of 25 patients converted from no recognition of the HPV 16 E6 protein to recognizing it.

Comparing the conversions from non-recognition of the HPV 16 E6 protein peptides to recognition of the peptides of that protein, it was 11 of 26 converted with PepCan versus only 4 of 25 with Candida extract alone (P=0.04).

Thus, including HPV 16 E6 peptides with the Candida extract in the vaccines increased immune response to those peptides (significantly), but decreased (though not significantly) clinical regression of the lesions. What that means is that the body knows better than we do. It is better to generate an increased global immune response by inducing a DTH response (whether that is with Candida extract or a topical immunosensitizer) and let the immune system figure out how best to attack HPV (or herpes virus or fungal or yeast organisms or cancer cells or other immune target), than to include in the vaccine HPV peptides or proteins that we think would be good targets and generate an immune response against those peptides or proteins. Our immune system has evolved to effectively fight cancer as well as infectious diseases, and we should just support it and let it figure out how to do their job rather than try to figure it out for the immune system. The present invention activates the immune system generally to stimulate it to do its job and fight cancer.

In the foregoing example, a clinical trial was conducted that showed that Candida extract injected intradermally to the skin of the arm or forearm was effective in causing regression of cervical intraepithelial neoplasia lesions (grade 2 or 3) (CIN 2/3). Candida extract injected into common warts has also been shown to be effective to cause regression of the warts. It is believed to act by causing a delayed-type hypersensitivity response because topical immunosensitizers, including squaric acid dibutylester (SADBE) also are effective in treating warts when topically applied to the wart and also cause a delayed-type hypersensitivity (DTH) response. (U.S. Pat. No. 6,350,451. Buckley, D. A. et al. 2001, The therapeutic use of topical contact sensitizers in benign dermatoses, Br. J. Dermatology 145:385-405. Lee et al., Journal of the American Academy of Dermatology, 1999, Vol. 41, pages 595-599.)

Importantly, in the clinical trial presented below in Example 1, Candida extract alone was more effective than a composition comprising Candida extract and human papilloma virus peptides. This suggests the efficacy is due solely to the non-specific DTH response caused by Candida extract, not a response or immunization targeted to specific HPV antigens. This is also suggested by results that a single SADBE application to the inner aspect of the upper arm was effective in preventing (reducing the number of) herpes labialis episodes in persons who were having frequent episodes. (U.S. Pat. Nos. 10,245,314 and 11,957,783, which are incorporated by reference.) In that case there was no application to a herpes lesion, again showing that the non-specific DTH response was effective.

In the clinical trial described below, a 0.5 ml dose of CANDIN (Candida extract) was more effective in causing regression of the CIN2/3 lesions than a 0.3 ml dose. A dose of just 0.1 ml of CANDIN is the standard dose for testing of immune competence.

The concentration of CANDIN is not defined by protein concentration or some easily measurable parameter but rather by observed effect on human test subjects. The package insert says “The skin-test strength of CANDIN® has been determined from dose-response studies in healthy adults (see CLINICAL STUDIES). The product is intended to elicit an induration response ≥5 mm in immunologically competent persons with cellular hypersensitivity to the antigen.” It is an advantage of SADBE and topical immunosensitizers that these are chemically defined and the strength of a dose is defined by the concentration of the topical immunosensitizer, not requiring a biological test.

Another advantage of topical immunosensitizers over intradermally injected recall antigens to treat HPV-caused cancers or pre-cancerous lesions is that one can conveniently make higher concentrations and stronger doses. CANDIN was more effective at a dose of 0.5 ml than 0.3 ml. The standard dose of CANDIN is just 0.1 ml and it would be probably not possible to inject more than 0.5 ml intradermally. In contrast, for a higher dose of a topical immunosensitizer, one could just use a higher concentration, with no practical limit on the concentration.

The dosing schedule of topical immunosensitizers can be from once per day to once per 6 months. To prevent oral herpes outbreaks it has been found that a single dose takes about 6 weeks to take effect, that is to alter the immune response to the herpes virus, and the effect of preventing outbreaks starts to taper off after three to four months, but still reduces the number of outbreaks compared to placebo for a full year. So in preferred embodiments of the present methods, patients are treated with a topical immunosensitizers once per month to once per four months. More frequent dosing is possible but typically sensitivity increases with more doses, so a rash with itching, pain, and redness at the application site can increase with further doses and more frequent doses.

The structure of squaric acid is shown below.

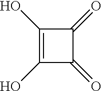

SADBE has the systemic name of 3,4-Dibutoxy-3-cyclobutene-1,2-dione. Its structure is shown below:

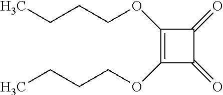 [SMILES: CCCCOc1c(OCCCC)c(=O)c1=O]

These contact sensitizers are available from several commercial sources, including Spectrum Chemicals & Laboratory Products, a division of Spectrum Chemical Manufacturing Corp., Gardena, California and New Brunswick, NJ.

In one embodiment of the methods the topical immunosensitizer is applied with a dermal patch. Suitable dermal patches are described in U.S. patent application Ser. No. 16/932,111, published as US 2021-0393542A1, which are incorporated by reference. The dermal patch can comprise a backing layer comprising a fabric overlaid by an adhesive over at least part of the area of the fabric; the backing layer overlaid over a portion of its area by an absorbent gauze layer; the absorbent gauze layer comprising a liquid or semi-liquid solution comprising a vehicle and a topical immunosensitizer dissolved in the vehicle. The solution can be applied to the patch by a patient by opening a vial of liquid immunosensitizer solution, dipping a cotton swab in the vial, and wetting the gauze of the dermal patch with the cotton swab, and then applying dermal patch to the patient's arm or elsewhere on his or her body.

It is another advantage of topical immunosensitizers over intradermal injection of Candida extract that patients can dose themselves at home with topical application immunosensitizers, which they would not be comfortable doing with intradermal injection.

The inventor has previously shown that SADBE works to prevent (reduce the number of) oral herpes episodes or cold sores by improving the immune response to the herpes simplex virus (HSV) that causes cold sores and in particular by increasing expression of interferon gamma (IFNG) by leukocytes when they encounter HSV. This is described in U.S. Pat. No. 11,957,783, which is incorporated by reference.

As described in U.S. Pat. No. 11,957,783, peripheral blood mononuclear cells (PBMCs) were collected from persons positive for IgG against herpes simplex virus type 1 (HSV-1) and having frequent, infrequent, or no herpes labialis outbreaks. The PBMCs were tested for proliferation against HSV-1 and immune gene expression in the presence of HSV-1. In addition, on day 1 after blood collection the subjects with frequent outbreaks were dosed topically on the arm once with squaric acid dibutyl ester (SADBE), which has been shown to reduce frequency of herpes labialis outbreaks, and their PBMCs were collected and tested 8 weeks later.

Those with good immune control of their HSV-1 infection (fewer or zero herpes labialis outbreaks) differed from those with poorer immune control (more outbreaks) in these ways:
Greater PBMC proliferation in vitro to HSV-1.
Higher expression of the IFNG gene and lower expression of the IL5 gene in PBMCs in vitro stimulated with HSV-1 virus.
Higher expression of almost all other immune related genes that are upregulated by stimulation with HSV-1 and lower expression of genes that are down-regulated.

Treatment of subjects with frequent outbreaks with a single topical dose of SADBE caused their PBMCs to become more like those of the subjects with infrequent or zero outbreaks 56 days after the a single topical treatment with SADBE in every one of these ways than they had been on day 1 before the treatment.

In particular, expression of interferon gamma (IFNG) by the isolated PBMCs when exposed to HSV increased 8 weeks after a topical application of SADBE to the patients' arms.

Interferon gamma expression was also increased in cells exposed to the entirely different stimulant of Candida extract, the same immunogen injected intradermally in Example 1 below. Candida is a yeast, so it has nothing in common with the HSV virus.

The data in Example 1 suggests that interferon gamma expression is also important for regression of pre-cancerous cervical lesions and therefore for control of HPV-caused cancers and by inference all cancers. Specifically, it was found that intradermal injection of Candida extract increased the proportion of patients with CD3 cells expressing IFNG against HPV peptides.

One embodiment of the invention involves a method of treating a mammal for a cancer comprising: applying a topical immunosensitizer to skin of the mammal.

In specific embodiments, the cancer is prostate cancer. Prostate cancer is often untreated and observed with watchful waiting or active monitoring until when and if progression requires a more radical treatment such as androgen deprivation therapy with a luteinizing hormone-releasing hormone (LHRH) agonist, such as leuprolide, or with surgical castration, or surgery to remove tumors or with radiation or chemotherapy. Topical immunosensitizer application can be used to treat prosate cancer, or any cancer, prior to, concurrently with, or after other cancer therapies such as chemotherapy, radiation, surgery, or immune checkpoint inhibitors. Or it can be used as a sole treatment where watchful waiting or active monitoring with no other active treatment is otherwise advisable and chosen by the patient's medical provider, as is often the case with prostate cancer.

In another specific embodiment of the methods, the cancer is low-grade non-Hodgkins lympoma. Low-grade non-Hodgkins lymphoma is incurable and slow-growing, so long-term treatment with harsh therapies like chemotherapy may be inadvisable, and topical immunosensitizer therapy, with its low side-effect profile of only a mild rash at the application site, and its suitability for self-dosing by the patient at home, avoiding time-consuming and costly trips to a medical facility for treatment, would often be appropriate.

The methods are suitable for preventing recurrence or progression of a cancer after a patient has been treated for the cancer and has a reduced tumor burden or no detectable tumor burden. The method acts by increasing immune response to the tumor cells, and that is more likely to be effective when the tumor burden is low. It is believed that we all constantly have microtumors developing and usually the immune system is able to eliminate those before they become detectable tumors.

Since the methods act by increasing anti-cancer activity of the immune system, they may be more effective in patients who are not immunocompromised by their cancer or by treatment, such as conventional chemotherapy. On the other hand, the methods may help somewhat to counteract the immunosupression effects of chemotherapy and thus may be suitable for use during chemotherapy.

The methods of the invention are particularly suitable for use in cancers associated with a weakened immune system, since the methods strengthen immune attack against cancer cells. These cancers associated with a weakened immune system include Kaposi's sarcoma and all blood cancers, including lymphomas, leukemias, and myelodysplastic syndrome. Thus, in specific embodiments the cancer is lymphoma, e.g., low grade, intermediate grade, or high grade non-Hodgkins lymphoma. In other specific embodiments the cancer is leukemia, e.g., acute myeloid leukemia, acute lymphocytic leukemia, chronic myeloid leukemia, or chronic lymphocytic leukemia. In another specific embodiment, the cancer is myelodysplastic syndrome.

Rationale for topical immunosensitizers efficacy to cause regression of pre-cancerous lesions in cancers caused by HPV and to treat cancers caused by HPV.
Intradermal injection of Candida causes regression of pre-cancerous HPV lesions.
Candida antigen acts by causing a delayed-type hypersensitivity response and it causes increased expression of interferon-gamma by immune cells when they encounter a foreign antigen (HPV). (Example 1)
Like Candida, topical immunosensitizers act by causing a delayed-type hypersensitivity response.
Like Candida, topical immunosensitizers cause increased expression of interferon gamma by immune cells when they encounter a foreign antigen (HSV and Candida). (U.S. Pat. No. 11,957,783 and McTavish H, Zerebiec K W, Zeller J C, Shekels L L, Matson M A, Kren B T. Immune characteristics correlating with HSV-1 immune control and effect of squaric acid dibutyl ester on immune characteristics of subjects with frequent herpes labialis episodes. Immun. Inflamm. Dis. 2019; 7 (1): 22-40.
Intradermal injection of Candida extract and topical application of topical immunosensitizers are both known to be effective to cause regression of common warts, which are caused by HPV. (U.S. Pat. No. 6,350,451. Buckley, D. A. et al. 2001, The therapeutic use of topical contact sensitizers in benign dermatoses. Br. J. Dermatology 145:385-405. Lee et al. Journal of the American Academy of Dermatology, 1999, Vol. 41, pages 595-599.)

Therefore one would accept that they are both equally effective to cause regression of pre-cancerous lesions caused by HPV and therefore to also treat HPV-caused cancers and prevent recurrence of HPV-caused cancers.

### Rationale to Prevent Recurrence of and Treat Cancers not Caused by HPV.

A single dose of a topical immunosensitizers (specifically SADBE) has been shown to broadly change the immune response against both herpes simplex virus and a fungal antigen (Candida extract), two totally unrelated organisms, from a type 2 immune response to a type 1 immune response and from a humoral or antibody immune response to a cellular or T-cell immune response. (U.S. Pat. No. 11,957,753, incorporated by reference. McTavish H, Zerebiec K W, Zeller J C, Shekels L L, Matson M A, Kren B T. Immune characteristics correlating with HSV-1 immune control and effect of squaric acid dibutyl ester on immune characteristics of subjects with frequent herpes labialis episodes. Immunity, Inflammation and Disease 2019; 7 (1): 22-40.)

With both types of these unrelated antigens it also causes an increase in expression of interferon gamma. The immune system is known to control cancer and usually kill the small number of cancerous cells that arise in our bodies all the time, preventing them from becoming tumors. Specifically, CD3+ cell expression of interferon gamma, which is shown here to be caused by Candida extract intradermal injection, and was previously shown to be caused by topical application of SADBE, activates killer T cells (CD8+ cells) which are known to kill tumor cells.

### Guidance

It has been shown that the efficacy of SADBE in preventing cold sore episodes begins to wane after 3 to 4 months, and that the effects of a SADBE dose take 2 to 8 weeks to take effect (much like a vaccine that takes a similar time frame for the immune system to respond and be trained). Accordingly, SADBE and other topical immunosensitizers can be applied topically approximately once per month to once per four months for efficacy to prevent recurrence of cancers.

It has been found for cold sore prevention that SADBE can be applied to the arm, and need not be applied to a herpes simplex virus (HSV) lesion. Likewise here it is shown that Candida works to cause regression of pre-cancerous HPV lesions when injected intradermally in the arm and there is no need to inject into a lesion or tumor.

The appropriate concentration of SADBE is approximately 2% or sufficient to cause a mild rash or delayed-type hypersensitivity response. Concentrations of about 0.1% to about 5% have been used and may be used in the methods of the invention. The appropriate concentration may vary from person to person. SADBE has been dissolved in dimethylsulfoxide (DMSO), and other topical immunosensitizers can be as well. Other suitable vehicles for the topical immunosensitizer include acetone, ethanol, isopropanol, butanol, and mixtures thereof and mixtures of any of these with DMSO.

Other suitable immunosensitizers include a squaric acid ester (e.g., squaric acid diethyl ester), diphenylcyclopropenone (DPCP), 1-chloro-2,4-dinitrobenzene (DNCB), 1-chloro-2,6-dinitrobenzene, and urushiol. Urushiol is the active ingredient in poison ivy that causes the skin rash. The appropriate concentrations of these other immunosensitizers for use in the invention are similar to those for SADBE.

### STATEMENTS OF THE INVENTION

1. A method of treating a cancer or precancerous lesions in a mammal comprising:
   applying a topical immunosensitizer to skin of the mammal;
   wherein the mammal is afflicted with a cancer caused by human papilloma virus (HPV) or precancerous lesions caused by HPV or was afflicted with said cancer and the cancer is now in remission or was afflicted with said precancerous lesions and the lesions have regressed or been removed.
1b. The method of statement 1 wherein the method reduces the probability of progression or recurrence of the cancer or progression of the precancerous lesions to cancer.
1c. The method of statement 1 wherein the cancer is in partial or complete remission.
2. The method of statement 1 wherein the mammal is a human.
3. The method of statement 1 wherein the method does not comprise applying the topical immunosensitizer to a cancerous or precancerous lesion.
3b. The method of statement 1 wherein the method comprises applying the topical immunosensitizer to intact skin that is not a site of an HPV lesion.
4. The method of statement 1 or 3b wherein the administering step comprises applying the topical immunosensitizer to skin on the inner aspect of the upper arm of the person.
5. The method of statement 1 wherein the topical immunosensitizer comprises a squaric acid ester, diphenylcyclopropenone (DPCP), 1-chloro-2,4-dinitrobenzene (DNCB), or 1-chloro-2,6-dinitrobenzene.
6. The method of statement 1 wherein the topical immunosensitizer comprises urushiol.
7. The method of statement 5 wherein the topical immunosensitizer comprises squaric acid dibutyl ester (SADBE).
8. The method of statement 7 wherein the topical immunosensitizer is SADBE and is applied as a solution in a solvent comprising DMSO.
9. The method of statement 1 wherein the human is afflicted with a cancer caused by HPV infection or was afflicted with said cancer and the cancer is now in remission.
9b. The method of statement 9 wherein the cancer is in partial remission.
9c. The method of statement 9 wherein the cancer is in complete remission.
10. The method of statement 1 wherein the cancer is cervical cancer, head and neck cancer, vulvar cancer, anal cancer, vaginal cancer, or penile cancer and the mammal is afflicted with said cancer or precancerous lesions at risk of progressing to said cancer.
11. The method of statement 10 wherein the cancer is cervical cancer.
12. The method of statement 1 wherein the mammal is afflicted with cancerous or precancerous cervical intraepithelial neoplasia lesions.
13. The method of statement 1 wherein the method is a method of preventing recurrence; wherein mammal was afflicted with said cancer and the cancer is now in remission or was afflicted with said precancerous lesions and the lesions have regressed or been removed.
13b. The method of statement 13 wherein the method reduces the probability of recurrence of the cancer or the precancerous lesions.
14. A method of treating a mammal for a cancer in remission comprising:
   applying a topical immunosensitizer to skin of the mammal;
   wherein the mammal has been diagnosed with a cancer, treated for the cancer, and the cancer is in remission at the time of the applying step.
14b. The method of statement 14 wherein the cancer is in partial or complete remission at the time of the applying step.
15. The method of statement 14 wherein the mammal is a human.
16. The method of statement 1 wherein the administering step comprises applying the topical immunosensitizer to skin on the inner aspect of the upper arm of the person.
17. The method of statement 14 wherein the cancer is a cancer commonly caused by human papilloma virus.
18. The method of statement 17 wherein the cancer is selected from the group consisting of cervical cancer, vulvar cancer, penile cancer, and head and neck cancer.
19. The method of statement 15 wherein the cancer is not a cancer caused by human papilloma virus.
20. The method of statement 19 wherein the cancer is breast cancer, colorectal cancer, gastric cancer, lymphoma, leukemia, myelodysplastic syndrome, glioblastoma, brain cancer, prostate cancer, lung cancer, testicular cancer, or a sarcoma.
21. The method of statement 14 wherein the topical immunosensitizer comprises a squaric acid ester, diphenylcyclopropenone (DPCP), 1-chloro-2,4-dinitrobenzene (DNCB), or 1-chloro-2,6-dinitrobenzene.
22. The method of statement 14 wherein the topical immunosensitizer comprises urushiol.
23. The method of statement 21 wherein the topical immunosensitizer comprises squaric acid dibutyl ester (SADBE).
24. The method of statement 23 wherein the topical immunosensitizer is SADBE and is applied as a solution in a solvent comprising DMSO.
25. A method of treating a mammal for cancer, the method comprising:
   applying a topical immunosensitizer to skin of the mammal; and administering to the mammal a checkpoint inhibitor;
   wherein the mammal has been diagnosed with a cancer and is in recognized need for treatment of the cancer.
26. The method of statement 25 wherein the checkpoint inhibitor is pembrolizumab (Keytruda), ipilimumab (Yervoy), nivolumab (Opdivo) or atezolizumab (Tecentriq).
27. The method of statement 25 wherein the cancer is lung cancer, liver cancer, breast cancer, melanoma, or urothelial cancer.
28. A method of treating a mammal for a cancer in remission comprising:
   applying a topical immunosensitizer to skin of the mammal.
29. The method of statement 28 wherein the mammal has been diagnosed with a cancer, treated for the cancer, and the cancer is in partial or complete remission at the time of the applying step.
30. The method of statement 29 wherein the cancer is in complete remission.
31. The method of statement 28 wherein the mammal at the time of the applying step is not immunocompromised from any anti-cancer treatments.
32. The method of statement 28 wherein the method further comprises treating the mammal with a further anti-cancer treatment that does not cause significant immunosuppression.
33. The method of statement 32 wherein the further anti-cancer treatment is selected from the group consisting of surgery, targeted radiation, a checkpoint inhibitor, and CAR-T therapy.
34. The method of statement 33 wherein the further anti-cancer treatment is selected from the group consisting of surgery and targeted radiation.
35. The method of statement 34 wherein the method further comprises treating the mammal with chemotherapy. The topical immunosensitizer may be applied between during chemotherapy (i.e., between chemotherapy doses or potentially during infusion of a dose) or after chemotherapy is completed (i.e., after the last planned dose of chemotherapy).
36. A method of treating a mammal for a cancer comprising:
   applying a topical immunosensitizer to skin of the mammal.
37. The method of statement 36 wherein the mammal has been diagnosed with a cancer, treated for the cancer, and the cancer is in partial or complete remission at the time of the applying step.
38. The method of statement 37 wherein the cancer is in complete remission at the time of the applying step.
39. The method of statement 36 wherein the mammal is a human.
40. The method of statement 39 wherein the cancer is prostate cancer.
41. The method of statement 39 wherein the cancer is low-grade non-Hodgkins lymphoma.
42. The method of statement 39 wherein the cancer is a lymphoma or a leukemia.
43. The method of statement 36 wherein the mammal at the time of the applying step is not immunocompromised from any anti-cancer treatments.
44. The method of statement 39 wherein the cancer is breast cancer, colorectal cancer, lung cancer, renal cell carcinoma, or endometrial cancer.
45. The method of statement 36 wherein the method further comprises treating the mammal with a further anti-cancer treatment that does not cause significant immunosuppression.
46. The method of statement 45 wherein the further anti-cancer treatment is selected from the group consisting of surgery, targeted radiation, and administration of a checkpoint inhibitor.
47. The method of statement 36 wherein the method further comprises treating the mammal with chemotherapy.
48. The method of statement 39 wherein the administering step comprises applying the topical immunosensitizer to skin on the inner aspect of the upper arm of the human.
49. The method of statement 36 wherein the topical immunosensitizer comprises a squaric acid ester, diphenylcyclopropenone (DPCP), 1-chloro-2,4-dinitrobenzene (DNCB), or 1-chloro-2,6-dinitrobenzene.
50. The method of statement 49 wherein the topical immunosensitizer comprises squaric acid dibutyl ester (SADBE).
51. The method of statement 49 wherein the topical immunosensitizer is applied as a solution in a solvent comprising DMSO.
52. The method of statement 36 wherein the human is afflicted with a cancer caused by HPV infection or was afflicted with said cancer and the cancer is now in remission.
53. The method of statement 36 wherein the cancer is cervical cancer, head and neck cancer, vulvar cancer, anal cancer, vaginal cancer, or penile cancer and the mammal is afflicted with said cancer or precancerous lesions at risk of progressing to said cancer.
54. The method of statement 36 wherein the method is a method of preventing recurrence of the cancer;
   wherein mammal was afflicted with said cancer and the cancer is now in remission.
55. The method of statement 36 wherein the method reduces the probability of progression or recurrence of the cancer.

### Advantages of the Embodiments of the Invention

Unlike most cancer treatments, the present methods of using topical application of a topical immunosensitizer to treat cancer or pre-cancerous conditions or to reduce recurrence, have these advantages:
They work with the body's natural immune system and strengethen it instead of weakening it.
They have low side effects, only a mild rash at the application site on the arm.
They are inexpensive. Topical immunosensitizers are small molecules that are inexpensive to manufacture.
They can be conveniently administered by patients themselves at home.

### Example 1

### Recall Antigen for Treating Cancerous and Precancerous Human Papilloma Virus Lesions

### SUMMARY

### Background

A non-surgical alternative for treating CIN2/3 is an unmet medical need for women of childbearing age due to a risk of cervical incompetency affecting subsequent pregnancies.

### Methods

PepCan consists of four human papillomavirus type 16 E6 peptides and a Candida skin testing reagent (adjuvant). In this randomized, double-blind Phase II study, women with biopsy-confirmed CIN2/3 were treated with PepCan or Candida one to one. Four intradermal injections were given 3 weeks apart followed by two visits 6 months apart. Histological responses were assessed using quadrant biopsies at the 12-month visit, and those whose lesions regressed to no CIN were considered to be complete responders. Regression rate of each treatment group was compared to that of a historical placebo group.

### Results

Of 99 subjects screened, 81 (81.8%) qualified for vaccination. With the intention-to-treat analysis, PepCan (n=39) showed 30.8% efficacy (95% confidence interval [CI], 17 to 47.6; p=0.25) while Candida (n=42) demonstrated 47.6% efficacy (95% CI, 32 to 63.6; p<0.001). Likewise, with the per-protocol analysis, PepCan (n=24) showed 45.8% efficacy (95% CI, 25.6 to 67.2; p=0.08) and Candida (n=29) showed 62.1% efficacy (95% CI, 42.3 to 79.3; p<0.001). There was no difference between efficacy of PepCan and Candida. No dose-limiting toxicity was observed.

### Conclusions

Only Candida was effective in inducing histological regression in the intention-to-treat and per-protocol analyses. PepCan and Candida treatments are safe. Candida could become a new treatment for CIN2/3 by eliciting general immune stimulation similar to checkpoint inhibitors. (Funded by the National Institutes of Health; ClinicalTrials.gov number, NCT02481414.)

### Introduction

Cervical intraepithelial neoplasia 2/3 (CIN2/3) is a precursor of cervical cancer which is the fourth most common cancer among women globally. The annual global incidence of cervical cancer is 604,127 cases and the mortality is 341,831 cases.^{1,2} It is mostly caused by human papillomavirus (HPV). HPV causes not only cervical cancer, but also anal, oropharyngeal, penile, vaginal, and vulvar cancers; it is estimated to be responsible for 5.2% of cancer cases in the world.^{3,4}

Standard surgical treatments of CIN2/3, such as loop electrical excision procedure (LEEP), are effective but may cause cervical incompetency resulting in doubling of preterm delivery rate,^{5,6} increased premature rupture of membrane,⁷ and lower birth weight.⁷ Therefore, the current treatment guidelines recommend 1-2 years of close observation in women, with cervical intraepithelial neoplasia 2 (CIN2), who are less than 25 years in age or who plan to have children. For cervical intraepithelial neoplasia 3 (CIN3), treatment is recommended but observation is an accepted option.⁶ Non-surgical alternatives which would leave the cervix anatomically intact such as therapeutic vaccines are unmet medical needs. As the World Health Organization has declared a call to action to accelerate elimination of cervical cancer through increasing prevention, detection, and treatment by 2030,⁸ the need for HPV therapeutic vaccines is great.

HPV transformation of squamous epithelium to a malignant phenotype is mediated by two early gene products, E6 and E7,⁹ and their expression is necessary for human papillomavirus type 16 (HPV 16) transformation of human cells (25-27).^{10,11} T-cell responses to HPV 16 E6 protein have been associated with favorable clinical outcomes such as viral clearance¹² and regression of cervical lesions.^{13,14} Therefore, the E6 protein is an especially attractive target for immunotherapy.

Traditionally, recall antigens, which typically include a panel of Candida, mumps, and Trichophyton, were used as controls to indicate intact cellular immunity when patients were being tested for Tuberculosis by placement of purified protein derivative (PPD) intradermally. A number of studies have demonstrated that recall antigen injections can also be used to treat common warts, ¹⁵⁻²⁰ and several studies have shown that treating warts with recall antigens is effective not only for injected warts but also distant untreated warts.^{15-19,21}. Furthermore, in vitro studies have demonstrated that Candida can induce interleukin-12 expression and T cell proliferation.^{22,23} Therefore, Candida was used for the first time as a vaccine adjuvant in PepCan in which four synthetic peptides covering HPV 16 E6 protein and Candida are combined in a Phase 1 clinical trial (ClinicalTrials.gov number, NCT01653249). The PepCan's safety was demonstrated, and the lowest dose turned out to be the most effective.^{24,25} In this report, we report the results of a Phase 2 clinical trial in which subjects with CIN2/3 were randomized to PepCan and Candida (NCT02481414).

### Methods

### Patients

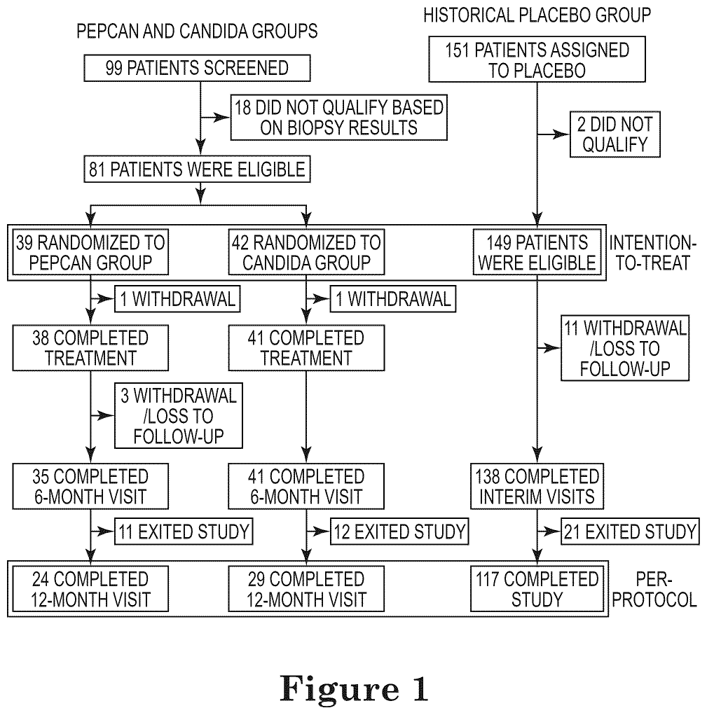

Patients (n=99) were screened between November 2015 and June 2021, and those with biopsy-proven CIN2/3 (n=81) were eligible for vaccination and were randomly assigned to the PepCan or Candida arm (FIG. 1). Exclusion criteria included a history of disease or treatment causing immunosuppression, pregnancy, breast feeding, allergy to Candida, a history of severe asthma, and a history of invasive squamous cell carcinoma of the cervix. A full descriptions of eligibility criteria are in the trial protocol submitted with this manuscript.

### Vaccine Composition and Delivery

The vaccine consisted of four current good manufacturing production-grade synthetic peptides covering the HPV 16 E6 protein [amino acid (aa) 1-aa45, aa46-aa80, aa81-aa115, and aa116-aa158] (CPC Scientific, San Jose, CA; Curia, Albany, NY).²³ Lyopholized peptides (50 microgram per peptide) were reconstituted with sterile water and were mixed with 0.3 ml of Candida (CANDIN, Nielsen Biosciences, San Diego, CA) in a syringe. The mixture was administered intradermally in any limbs, but most often in forearms.

### Trial Design and End Points

This clinical trial was a randomized double-blind Phase 2 study in which patients were assigned to PepCan or Candida arm at a ratio of one to one. Four injections were given 3 weeks apart, and the subjects return for observational visit 6 months and 12 months after the last injection (data not shown). A liquid-based cytology samples (Thin-Prep, Hologic, Marlborough, MA) were collected for HPV-DNA testing for 37 individual HPV types (Linear Array HPV Genotyping Test, Roche Molecular Diagnostics, Pleasanton, CA) at screening visit, 6-month visit, and 12-month visit. HPV 16 viral load was also determined^{24,26} for subjects who were HPV 16-positive at the screening visit. The primary endpoint was histological regression, which was assessed using quadrant biopsies, collected at the 12-month visit, examined by two pathologists blinded to each other's diagnosis. For patients who exited the study after 6-month visit, the results of biopsy and/or LEEP were used to assess histological responses. The secondary endpoint was safety, which was assessed from the time informed consent was obtained until the 6- or 12-month visit using version the National Cancer Institute Common Terminology Criteria for Adverse Events version 4.03. A patient with no dysplasia was considered to be a complete responder, a patient with cervical intraepithelial neoplasia 1 (CIN1) was considered to be a partial responder, and a patient with CIN2/3 was considered to be a non-responder. Only patients with no CIN were considered to be histological responders with a stringent criterion, and those with no CIN and CIN1 were considered to be responders with a lenient criterion.

Additional endpoints involved examination of peripheral immune responses by examining T cell activity to the HPV 16 E6 protein using an interferon-gamma enzyme-linked immunospot (ELISPOT) assay. Immune profiling measuring T-helper type 1 (Th1), T-helper type 2 (Th2), regulatory T cells (Tregs),^{24,25} and myeloid-derived suppressor cells (MDSCs)^{27,28} was also performed using fluorescent-activated cell sorter (FACS) with blood samples drawn at visit 1, visit 3, 6-month visit, and 12-month visit. In search for a potential biomarker predictive for vaccine response, pre-vaccination samples were examined for plasma cytokines, plasma metabolomics, and cervical microbiome. Low resolution class I and class II HLA typing was performed using DNA from any remaining blood cells.

### Trial Oversight

The study was approved by the Institutional Review Board, and was supported by the National Cancer Institute. A written informed consent was obtained from each participant. No commercial support was provided for this study, and no person who is not an author contributed to the manuscript. All authors participated in data aquisition, had access to data, wrote and/or edited the manuscript, and vouched for the accuracy and completeness of the data.

### Statistical Analysis

The sample size was calculated assuming 60% response rate for the PepCan group. In comparison, the historical placebo group reported 29% (34 of 117) response rate.² It was estimated that a sample size of 35 would provide 93% power to test the null hypothesis that π=0.29 (historical control placebo rate) and detect a difference of 0.31 with a two-sided type I error of 5%. The goal was to start vaccinating 40 patients in each treatment group to account for attrition. The study was not powered to show the differences between the Candida and placebo groups and between the PepCan and Candida groups. Each treatment group was compared to the historical placebo group using the exact binomial test (two-sided). The PepCan and Candida groups were compared using Pearson's χ2-tests (two-sided). The Clopper-Pearson method was used to determine the 95% confidence intervals.

### Results

### Patients and Treatment

Thirty-nine patients were randomly assigned to the PepCan arm and 42 patients were assigned to the Candida arm (the intention-to-treat population), and their baseline characteristics were well balanced (Table 1). Thirty-eight and 42 patients in PepCan and Candida groups respectively completed treatment, 35 patients and 41 patients completed the 6-month visit, and 24 patients and 29 patients completed the 12-month visit (FIG. 1).

### Efficacy

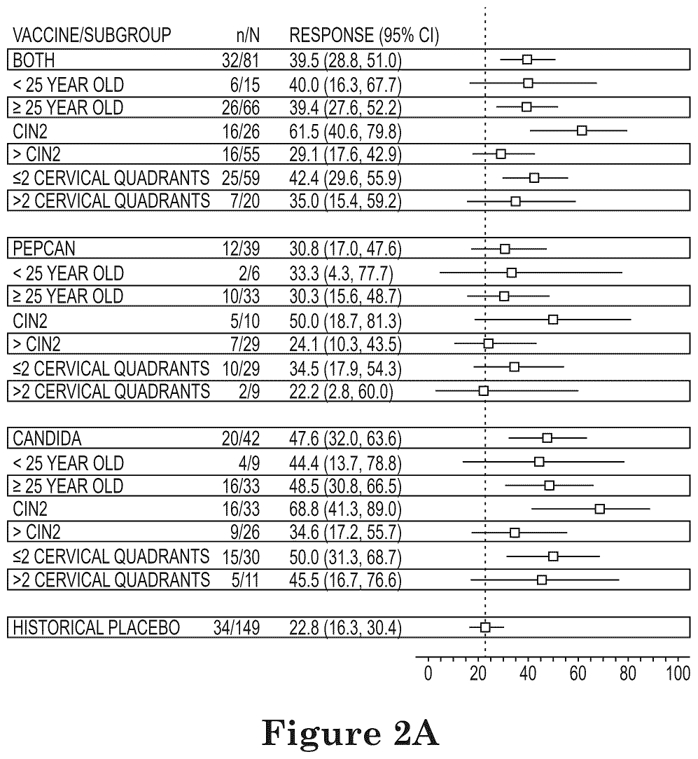

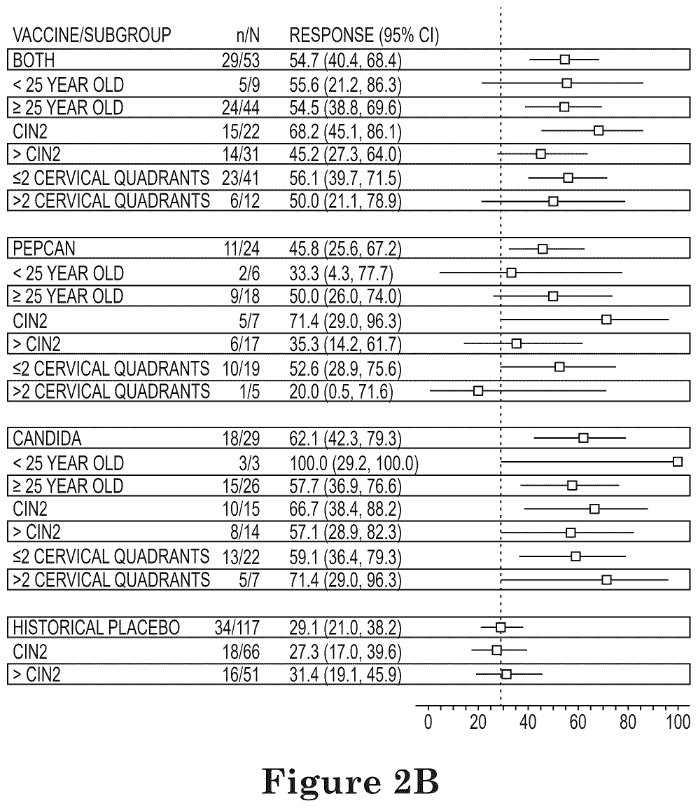

In comparison to historical placebo group, the Candida group, but not the PepCan group, demonstrated significantly higher histological regression rates in the intention-to-treat (47.6%, 95% CI, 32 to 63.6; p<0.001) and per-protocol (62.1%, 95% CI, 42.3 to 79.3; p<0.001) populations. There were no significant differences in histological response rates of the PepCan and Candida groups in both analyses. So, both groups combined and separately were analyzed for stringent histological responses in the intention-to-treat (FIG. 2A) and per-protocol populations (FIG. 2B) as well as in subgroups characterized by age, initial diagnosis, and initial number of cervical quadrants involved. Analogous analyses were performed using the lenient criterion for histological response (data not shown). No significant differences were found between the subgroups examined except with the comparisons of initial diagnoses between “CIN2” and “>CIN2”. In the intention-to-treat population for both treatment groups combined, “CIN2” had response rate of 61.5% and “>CIN2” had that of 29.1% (relative risk [RR], 2.12; 95% CI, 1.26 to 3.53; p=0.01) using the stringent criterion. With the lenient criterion, the response rate for “CIN2” was 73.1% and that of “>CIN2” was 36.4% (RR, 2.01; 95% CI, 1.31 to 3.08; p=0.004) with both groups, the response rate for “CIN2” was 87.5% and that of “>CIN2” was 38.5% (RR, 2.28, 95% CI, 1.39 to 3.99; p=0.003) for the Candida group.

### Safety

Treatment-related commonly occurring (with 5% or more of injections) adverse events (AEs) are summarized in Tables 1. No treatment-related dose-limiting toxicity (DLT) was reported, and the most frequent AE was immediate injection site reaction for both treatment groups. Fever (RR, 3.60, 95% CI, 1.01 to 12.82; p=0.05) and myalgia (RR 3.99, 95% CI, 2.06 to 7.75; p<0.001) were significantly more frequently observed in the PepCan group in comparison to the Candida group (Table 1). No patients discontinued due to AEs.

### Immune Responses

New CD3 T-cell responses, following vaccinations, to at least one region of the HPV 16 E6 protein were detected in 18 of 33 patients (54.5%) in the PepCan group and in 17 of 38 (44.7%) patients in the Candida group. Anti-HPV 16 E6 T cell responses were detectable prior to vaccination in 7 of 33 patients (21.2%) in the PepCan group and in 13 of 38 patients (34.2%) in the Candida group. These differences and the differences between histological responders and non-responders using the stringent criterion were not statistically significant. But the increase in proportion of patients with CD3 cells expressing IFNG against these selected HPV peptides from before vaccination to after vaccination, went from 20 of 71 to 35 of 71, which was significant (P=0.02).

The FACS analysis of peripheral immune cells revealed that the percentage of Tregs was higher in histological non-responders in comparison to responders (stringent) prior to vaccination and at the 12-month visit; however, the comparisons were no longer significant after adjustments for multiple analyses (data not shown). No changes following after vaccinations that were consistent and significant after adjustments for multiple analyses were found (data not shown).

### HPV

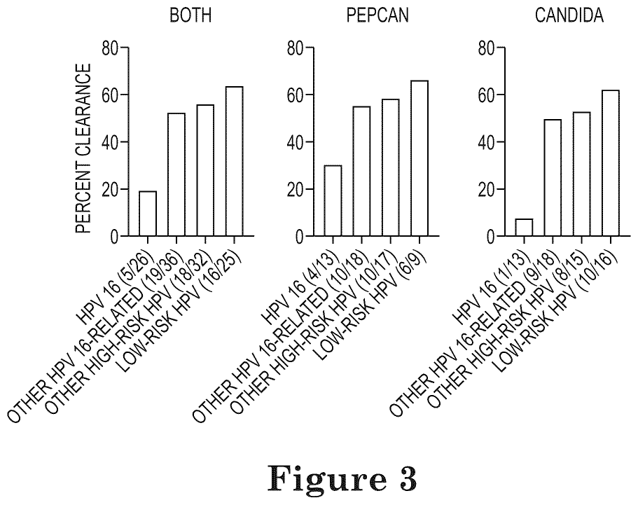

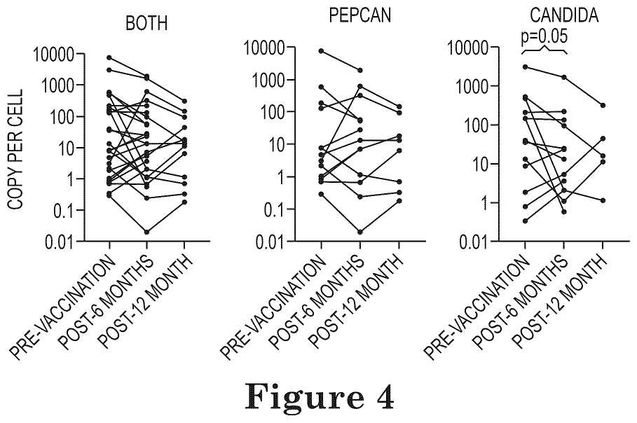

When clearance of HPV infection at exit visit (6- or 12-month visit) by risk groups was examined, no significant differences were found between the PepCan and Candida group (FIG. 3). For patients who were HPV 16-positive at the screening visit, the HPV 16 viral load was examined. A trend for HPV 16 viral load decreasing at the 6-month visit was observed in the Candida group, suggesting the HPV antigens may not be necessary for reducing viral loads (FIG. 4; p=0.05).

### Biomarkers

Plasma cytokines and plasma metabolomics in pre-vaccination samples were examined for their correlations with histological response (stringent) for patients who completed 6-month visit. None of the significant associations remained after adjustments for multiple comparisons (data not shown). After examining the cervical microbiome, there were no differences in diversity metrices (data not shown) between histological responders and non-responders (stringent). No individual taxa were associated with histological responses either (data not shown). Likewise, there were no significant association with HLA types with histological responses (stringent) after correcting for multiple comparisons (data not shown). However, some HLA types were found at significantly higher proportions in the patient population including HLA B14, B15, B40, B49, C03, DR03, and DQ03. In particular, DQ03 was found to be nearly ten times more frequent in the patient population in comparison to the United States population. The increased frequencies of HLA B14, B15, B40, C03, DR03, and DQ03 were also observed in our Phase I study.25 In particular, the increased risk for cervical neoplasia in people with DQ03 has also been reported by others. 29-31

### Discussion

CIN2/3 is a premalignant lesion of the uterine cervix that is known to be high risk for progression to malignancy in comparison to its lower grade counterpart, cervical intraepithelial neoplasia 1 (CIN1). While CIN2/3 lesions are usually treated (but not CIN1) with surgical resection such as LEEP, observation is desirable for women younger than 25 and women who are planning to become pregnant due to possible side effects which could result in cervical incompetency.⁶ The availability of an effective HPV therapeutic vaccine that would promote regression of CIN without affecting the cervical anatomy is an unmet need; however, none has been approved by the Food and Drug Administration to date although many different versions have been tested in clinical trials.³²⁻³⁷

In this study, the effectiveness of PepCan and Candida were examined. Unexpectedly, only Candida was significantly more effective in regressing CIN2/3 in comparison to a historical placebo group.² Given that PepCan with higher amounts of HPV 16 E6 peptides were less effective in regressing CIN2/3 and in mounting anti-HPV T cell responses in our Phase 1 study,^{24,25} may be possible that the HPV peptides in this formulation may have some inhibitory effect. While Candida is not significantly more effective than PepCan, it is clear that addition of the peptides does not contribute to the vaccine effectiveness. These results demonstrate that the adjuvant effect is more important than the HPV antigens for therapeutic vaccines which are given to patients who already been infected.

Safety of PepCan and Candida has been demonstrated as there were not DLTs reported during the study. Interestingly, fever and myalgia were more frequently reported in the PepCan group suggesting that it was cause by the HPV peptides.

As demonstrated in our Phase I study,^{24,25} the presence of new T cell responses to HPV 16 E6 after vaccination was not correlated with the histological responses possibly because peripheral T cell responses were examined. While it is technically possible to demonstrate the presence of vaccine-induced T cell responses in the cervix,³⁸ applying it broadly in a clinical trial is still a challenge.

In vitro, Candida has been shown to induce T cell proliferation and secretion of IL-12.^{23,39} However, we did not observe increase in peripheral Th1 cells in this study which we did in the Phase I study.^{24,25}

As we have demonstrated efficacy of Candida for treating CIN2/3 in comparison to a historical placebo group, the next step would be to perform a Phase 3 study with a concurrent placebo group. However, risk of disease progression in the placebo group, and the reluctance of patients to enroll when other treatments are available would be of concern. Another alternative is to explore whether Candida could be more potent by increasing the volume and/or the number of injections. A multi-center Phase 2 study using Candida to treat common warts has demonstrated 65.9% efficacy (29/44) at the 0.3 mL dose and 79.5% efficacy (31/39) at the 0.5 mL dose in regressing the largest injected wart.⁴⁰ This is a 21% increase in efficacy. Therefore, it may be possible to examine Candida as an alternative to standard surgical treatments in a non-inferiority trial. This would possibly make Candida a first-line option for treating CIN2/3.

### Tables

TABLE 1
Baseline Characteristics of the Patients\hat{ }

| | Group | | |
|---|---|---|---|
| | PepCan | Candida | |
| | Group | Group | Total |
| Characteristic | N = 39 | N = 42 | N = 81 |
| Age, years | | | |
| Mean ± SD | 31.3 ± 6.4 | 31.4 ± 6.1 | 31.3 ± 6.2 |
| Range | 21.5-45.1 | 22.4-43.6 | 21.5-45.1 |
| <25 years - no. (%) | 6 (15.4) | 9 (21.4) | 15 (18.5) |
| ≥25 years - no. (%) | 33 (84.6) | 33 (78.6) | 66 (81.5) |
| Race - no. (%) | | | |
| Caucasian | 34 (87.2) | 37 (88.1) | 71 (87.7) |
| African American | 4 (10.3) | 4 (9.5) | 8 (9.9) |
| Other | 1 (2.6) | 1 (2.4) | 2 (2.5) |
| Ethnicity - no. (%) | | | |
| Hispanic | 11 (28.2) | 12 (28.6) | 23 (28.4) |
| Non-Hispanic | 28 (71.8) | 30 (71.4) | 58 (71.6) |
| >CIN2 - no. (%) | 29 (74.4) | 26 (61.9) | 55 (67.9) |
| =CIN2 - no. (%) | 10 (25.6) | 16 (38.1) | 26 (32.1) |
| No. cervical quadrants | | | |
| Mean ± SD | 1.89 ± 1.06 | 2.05 ± 1.02 | 1.97 ± 1.04 |
| >2 quadrants - no. (%) | 9 (23.7) | 11 (26.8) | 20 (25.3) |
| ≤2 quadrants - no. (%) | 29 (76.3) | 30 (73.2) | 59 (74.7) |
| Body mass index (kg/m²) | | | |
| Mean ± SD | 27.3 ± 6.2 | 29.1 ± 7.3 | 28.2 ± 6.8 |
| ≥25 kg/m² - no. (%) | 21 (53.8) | 26 (63.4) | 47 (58.8) |
| ≥30 kg/m² - no. (%) | 11 (28.2) | 19 (46.3) | 30 (37.5) |
| Albumin <3.5 g/dL - no. (%) | 1 (2.6) | 3 (7.3) | 4 (5.0) |
| Total protein <6.4 | 3 (7.7) | 5 (12.2) | 8 (10.0) |
| g/dL - no. (%) | | | |
| HPV prophylactic | 7 (17.9) | 7 (16.7) | 14 (17.3) |
| vaccine - no. (%) | | | |
| Any college - no. (%) | 20 (52.6) | 20 (47.6) | 40 (50.0) |
| Any children - no. (%) | 27 (69.2) | 27 (64.3) | 54 (66.7) |
| ≥5 sexual partners - no. (%) | 19 (48.7) | 30 (73.2) | 49 (61.3) |
| Ever smoked - no. (%) | 18 (46.1) | 15 (35.7) | 33 (40.7) |
| Currently smoke - no. (%) | 8 (20.5) | 9 (21.4) | 17 (21.0) |
| Ever used oral | 27 (69.2) | 32 (76.2) | 59 (72.8) |
| contraceptives - no. (%) | | | |
| Currently use oral | 6 (15.4) | 15 (35.7) | 21 (25.9) |
| contraceptives - no. (%) | | | |

\hat{ }Data for the intention-to-treat population are shown which included patients who qualified for vaccination and were randomized.

TABLE 2
A comparison of injection-related adverse events occurring
in ≥5% of injections with PepCan or Candida

| | PepCan n = 152 | | | | | | Candida n = 164 | | | | | |
|---|---|---|---|---|---|---|---|---|---|---|---|---|
| | Grade 1 | | Grade 2 | | All Grades | | Grade 1 | | Grade 2 | | All Grades | |
| | n (%) | | n (%) | | n (%) | | n (%) | | n (%) | | n (%) | |
| Fever | 9 | (5.9%) | 1 | (0.7%) | 10 | (6.6%) | 3 | (1.8%) | 0 | (0.0%) | 3 | (1.8%) |
| Headache | 13 | (8.6%) | 4 | (2.6%) | 17 | (11.2%) | 15 | (9.2%) | 3 | (1.8%) | 18 | (11.0%) |
| Injection site | 63 | (41.5%) | 25 | (16.5%) | 88 | (57.9%) | 58 | (35.4%) | 31 | (18.9%) | 89 | (54.3%) |
| reaction, <24 h | | | | | | | | | | | | |
| Injection site | 23 | (15.1%) | 31 | (20.4%) | 54 | (35.5%) | 39 | (23.8%) | 18 | (11.0%) | 57 | (34.8%) |
| reaction, ≥24 h | | | | | | | | | | | | |
| Myalgia | 35 | (23.0%) | 2 | (1.3%) | 37 | (24.3%) | 9 | (5.5%) | 1 | (0.6%) | 10 | (6.1%) |
| Nausea | 19 | (12.5%) | 0 | (0.0%) | 19 | (12.5%) | 18 | (11.0%) | 0 | (0.0%) | 18 | (11.0%) |

n, number of injections

### REFERENCES

1. Cancer IAfRo. GLOBOCAN 2020 CANCER FACT SHEET. (https://gco.iarc.fr/today/online-analysis-multi-bars?v=2020&mode=cancer&mode_population=countries&population=900&populations=900& key=total&sex=2&cancer=39&type=0&statistic=5&prevalence=0&population_group=0&ages group % 5B %5D=0&ages_group % 5B %5D=17&nb_items=10&group_cancer=1&include_nmsc=0&include_nmsc_other=1&type_multiple=% 257B %2522inc %2522%253Atrue %252C %2522mo rt % 2522%253Atrue %252C %2522prev %2522%253Afalse %257D&orientation-horizontal&type_sort=0&type_nb_items=% 257B %2522top %2522%253Atrue %252C %2522bottom %2522%253 Afalse % 257D.
2. Meyskens F L, Jr., Surwit E, Moon T E, et al. Enhancement of regression of cervical intraepithelial neoplasia II (moderate dysplasia) with topically applied all-trans-retinoic acid: a randomized trial. J Natl Cancer Inst 1994; 86 (7): 539-43. (In eng) (http://www.ncbi.nlm.nih.gov/pubmed/8133537).
3. Chaturvedi AK. Beyond cervical cancer: burden of other HPV-related cancers among men and women. J Adolesc Health 2010; 46 (4 Suppl): S20-6. (In eng). DOI: S1054-139X (10) 00076-5 [pii] 10.1016/j.jadohealth.2010.01.016.
4. Tota J E, Chevarie-Davis M, Richardson L A, Devries M, Franco EL. Epidemiology and burden of HPV infection and related diseases: implications for prevention strategies. Prev Med 2011; 53 Suppl 1: S12-21. (In eng). DOI: 10.1016/j.ypmed.2011.08.017S0091-7435 (11) 00306-9 [pii].
5. Bruinsma F J, Quinn M A. The risk of preterm birth following treatment for precancerous changes in the cervix: a systematic review and meta-analysis. BJOG 2011; 118 (9): 1031-41. (In eng). DOI: 10.1111/j.1471-0528.2011.02944.x.
6. Massad L S, Einstein MH, Huh W K, et al. 2012 updated consensus guidelines for the management of abnormal cervical cancer screening tests and cancer precursors. Obstet Gynecol 2013; 121 (4): 829-46. (In eng). DOI: 10.1097/AOG.0b013e3182883a3400006250-201304000-00019 [pii].
7. Zhuang H, Hong S, Zheng L, et al. Effects of cervical conisation on pregnancy outcome: a meta-analysis. J Obstet Gynaecol 2019; 39 (1): 74-81. DOI: 10.1080/01443615.2018.1463206.
8. Organization WH. WHO Director-General calls for all countries to take action to help end the suffering caused by cervical cancer. (https://www.who.int/news/item/18-05-2018-who-dg-calls-for-all-countries-to-take-action-to-help-end-the-suffering-caused-by-cervical-cancer).
9. Kumar V, Fausto N, Abbas A. Cervix. Robbins & Cotran Pathologic Basis of Disease. Seventh ed2004: 1072-1079.
10. Schlegel R, Phelps WC, Zhang Y L, Barbosa M. Quantitative keratinocyte assay detects two biological activities of human papillomavirus DNA and identifies viral types associated with cervical carcinoma. Embo J 1988; 7 (10): 3181-7.
11. Pirisi L, Yasumoto S, Feller M, Doniger J, DiPaolo J. Transformation of human fibroblasts and keratinocytes with human papillomavirus type 16 DNA. J Virol 1987; 61:1061-1066.
12. Nakagawa M, Stites D P, Patel S, et al. Persistence of human papillomavirus type 16 infection is associated with lack of cytotoxic T lymphocyte response to the E6 antigens. J Infect Dis 2000; 182 (2): 595-8. (In eng). DOI: JID000053 [pii] 10.1086/315706.
13. Kim K H, Greenfield W W, Cannon M J, Coleman H N, Spencer H J, Nakagawa M. CD4+ T-cell response against human papillomavirus type 16 E6 protein is associated with a favorable clinical trend. Cancer Immunol Immunother 2012; 61 (1): 63-70. (In eng). DOI: 10.1007/s00262-011-1092-5.
14. Nakagawa M, Gupta S K, Coleman H N, Sellers M A, Banken J A, Greenfield W W. A favorable clinical trend is associated with CD8 T-cell immune responses to the human papillomavirus type 16 e6 antigens in women being studied for abnormal pap smear results. J Low Genit Tract Dis 2010; 14 (2): 124-9. (In eng). DOI: 10.1097/LGT.0b013e3181c6f01e00128360-201004000-00008 [pii].
15. Clifton M M, Johnson S M, Roberson P K, Kincannon J, Horn T D. Immunotherapy for recalcitrant warts in children using intralesional mumps or Candida antigens. Pediatr Dermatol 2003; 20 (3): 268-71. (In eng). DOI: 20318 [pii].
16. Horn T D, Johnson S M, Helm R M, Roberson P K. Intralesional immunotherapy of warts with mumps, Candida, and Trichophyton skin test antigens: a single-blinded, randomized, and controlled trial. Arch Dermatol 2005; 141 (5): 589-94. (In eng). DOI: 141/5/589 [pii] 10.1001/archderm. 141.5.589.
17. Johnson S M, Horn T D. Intralesional immunotherapy for warts using a combination of skin test antigens: a safe and effective therapy. J Drugs Dermatol 2004; 3 (3): 263-5. (In eng) (http://www.ncbi.nlm.nih.gov/pubmed/15176159).
18. Johnson S M, Roberson P K, Horn T D. Intralesional injection of mumps or Candida skin test antigens: a novel immunotherapy for warts. Arch Dermatol 2001; 137 (4): 451-5. (In eng). DOI: dst00085 [pii].
19. Phillips R C, Ruhl T S, Pfenninger J L, Garber M R. Treatment of warts with Candida antigen injection. Arch Dermatol 2000; 136 (10): 1274-5. (In eng). DOI: dlt1000-7 [pii].
20. Maronn M, Salm C, Lyon V, Galbraith S. One-year experience with candida antigen immunotherapy for warts and molluscum. Pediatr Dermatol 2008; 25 (2): 189-92. (In eng). DOI: PDE630 [pii] 10.1111/j.1525-1470.2008.00630.x.
21. Kim K H, Horn T D, Pharis J, et al. Phase 1 clinical trial of intralesional injection of Candida antigen for the treatment of warts. Arch Dermatol 2010; 146 (12): 1431-3. (In eng). DOI: 146/12/1431 [pii] 10.1001/archdermatol.2010.350.
22. Nakagawa M, Daniels J, Goodwin A, Nagarajan U. Use of Candida Extract as a Novel Vaccine Adjuvant. Immunotherapy & Vaccine Summit. Cambridge: Cambridge Healthtech Institute; 2010.
23. Wang X, Coleman H N, Nagarajan U, Spencer H J, Nakagawa M. Candida skin test reagent as a novel adjuvant for a human papillomavirus peptide-based therapeutic vaccine. Vaccine 2013; 31 (49): 5806-13. (In eng). DOI: 10.1016/j.vaccine.2013.10.014S0264-410X (13) 01366-2 [pii].
24. Coleman H N, Greenfield W W, Stratton S L, et al. Human papillomavirus type 16 viral load is decreased following a therapeutic vaccination. Cancer Immunol Immunother 2016; 65 (5): 563-73. (In eng). DOI: 10.1007/s00262-016-1821-x10.1007/s00262-016-1821-x [pii].
25. Greenfield W W, Stratton S L, Myrick R S, et al. A phase I dose-escalation clinical trial of a peptide-based human papillomavirus therapeutic vaccine with skin test reagent as a novel vaccine adjuvant for treating women with biopsy-proven cervical intraepithelial neoplasia 2/3. Oncoimmunology 2015; 4 (10): e 1031439. (In Eng). DOI: 10.1080/2162402X.2015.10314391031439 [pii].
26. Mirabello L, Schiffman M, Ghosh A, et al. Elevated methylation of HPV16 DNA is associated with the development of high grade cervical intraepithelial neoplasia. Int J Cancer 2013; 132 (6): 1412-22. (In eng). DOI: 10.1002/ijc.27750.
27. Filipazzi P, Valenti R, Huber V, et al. Identification of a new subset of myeloid suppressor cells in peripheral blood of melanoma patients with modulation by a granulocyte-macrophage colony-stimulation factor-based antitumor vaccine. J Clin Oncol 2007; 25 (18): 2546-53. DOI: 10.1200/JCO.2006.08.5829.
28. Poschke I, Mougiakakos D, Hansson J, Masucci G V, Kiessling R. Immature immunosuppressive CD14+HLA-D R-/low cells in melanoma patients are Stat3hi and overexpress CD80, CD83, and D C-sign. Cancer Res 2010; 70 (11): 4335-45. DOI: 10.1158/0008-5472.CAN-09-3767.
29. Hildesheim A, Schiffman M, Scott D R, et al. Human leukocyte antigen class I/I I alleles and development of human papillomavirus-related cervical neoplasia: results from a case-control study conducted in the United States. Cancer Epidemiol Biomarkers Prev 1998; 7 (11): 1035-41. (In eng) (http://www.ncbi.nlm.nih.gov/pubmed/9829713).
30. Madeleine M M, Johnson L G, Smith A G, et al. Comprehensive analysis of HLA-A, HLA-B, HLA-C, HLA-DRB1, and HLA-DQB1 loci and squamous cell cervical cancer risk. Cancer Res 2008; 68 (9): 3532-9. (In eng). DOI: 10.1158/0008-5472.CAN-07-647168/9/3532 [pii].
31. Wang S S, Wheeler C M, Hildesheim A, et al. Human leukocyte antigen class I and I I alleles and risk of cervical neoplasia: results from a population-based study in Costa Rica. J Infect Dis 2001; 184 (10): 1310-4. (In eng). DOI: JID010476 [pii] 10.1086/324209.
32. Choi Y J, Hur S Y, Kim T J, et al. A Phase I I, Prospective, Randomized, Multicenter, Open-Label Study of GX-188E, an HPV DNA Vaccine, in Patients with Cervical Intraepithelial Neoplasia 3. Clin Cancer Res 2020; 26 (7): 1616-1623. DOI: 10.1158/1078-0432.CCR-19-1513.
33. Einstein M H, Kadish A S, Burk R D, et al. Heat shock fusion protein-based immunotherapy for treatment of cervical intraepithelial neoplasia III. Gynecol Oncol 2007; 106 (3): 453-60. DOI: 10.1016/j.ygyno.2007.04.038.
34. Garcia-Hernandez E, Gonzalez-Sanchez J L, Andrade-Manzano A, et al. Regression of papilloma high-grade lesions (CIN 2 and CIN 3) is stimulated by therapeutic vaccination with MVA E2 recombinant vaccine. Cancer Gene Ther 2006; 13 (6): 592-7. (In eng). DOI: 7700937 [pii] 10.1038/sj.cgt.7700937.
35. Harper D M, Nieminen P, Donders G, et al. The efficacy and safety of Tipapkinogen Sovacivec therapeutic HPV vaccine in cervical intraepithelial neoplasia grades 2 and 3: Randomized controlled phase I I trial with 2.5 years of follow-up. Gynecol Oncol 2019; 153 (3): 521-529. DOI: 10.1016/j.ygyno.2019.03.250.
36. Ikeda Y, Adachi K, Tomio K, et al. A Placebo-Controlled, Double-Blind Randomized (Phase IIB) Trial of Oral Administration with HPV16 E7-Expressing Lactobacillus, GLBL101c, for the Treatment of Cervical Intraepithelial Neoplasia Grade 2 (CIN2). Vaccines (Basel) 2021; 9 (4). DOI: 10.3390/vaccines9040329.
37. Trimble C L, Morrow M P, Kraynyak K A, et al. Safety, efficacy, and immunogenicity of VGX-3100, a therapeutic synthetic DNA vaccine targeting human papillomavirus 16 and 18 E6 and E7 proteins for cervical intraepithelial neoplasia 2/3: a randomised, double-blind, placebo-controlled phase 2b trial. Lancet 2015 (In Eng). DOI: S0140-6736 (15) 00239-1 [pii] 10.1016/S0140-6736 (15) 00239-1.
38. Shibata T, Shah S, Evans T, et al. Expansion of Human Papillomavirus-Specific T Cells in Periphery and Cervix in a Therapeutic Vaccine Recipient Whose Cervical High-Grade Squamous Intraepithelial Lesion Regressed. Front Immunol 2021; 12:645299. DOI: 10.3389/fimmu.2021.645299.
39. Nakagawa M, Coleman H N, Wang X, Daniels J, Sikes J, Nagarajan U M. IL-12 secretion by Langerhans cells stimulated with Candida skin test reagent is mediated by dectin-1 in some healthy individuals. Cytokine 2014; 65 (2): 202-9. (In eng). DOI: 10.1016/j.cyto.2013.11.002S1043-4666 (13) 00752-7 [pii].
40. ClinicalTrials.gov. Safety and Efficacy of Varying Regimens of CANDIN for Treatment of Common Warts (Verruca Vulgaris) ClinicalTrials.gov I D NCT02393417. (https://clinicaltrials.gov/study/NCT02393417?intr=CANDIN&cond=wart&term=Phase % 202&r ank=1).

### Example 2

### Prophetic Example of Prevention of Recurrence of Cervical Cancer

### Design:

Patients with cervical cancer after completing treatment with partial or complete remission are recruited and randomized to receive placebo topical application of SADBE dissolved in dimethylsulfoxide (DMSO) at a concentration of 1% to 2% (weight to volume) or placebo (DMSO alone). The SADBE solution is administered by wetting the gauze portion of a dermal patch with the SADBE solution (US Patent Application 16/932,111, publication 2021-0393542A1, which is incorporated by reference) and applying the dermal patch to the inner aspect of the upper arm (the surface facing the chest) and wearing the patch for 2 to 6 hours. Patients are dosed once every three months.

Patients will be eligible for enrollment following any treatment for cervical cancer, including surgery, chemotherapy, and radiation.

### Results:

Patients receiving the SADBE solution are significantly less likely to have recurrence or progression of cervical cancer within 12 months study entry than patients receiving placebo.

### Example 3

### Prophetic Example of Prevention of Recurrence of Cancer not Caused by Human Papillomavirus

### Design:

Patients with a cancer not caused by HPV after completing treatment with partial or complete remission are recruited and randomized to receive topical application of SADBE dissolved in dimethylsulfoxide (DMSO) at a concentration of 1% to 2% (weight to volume) or placebo (DMSO alone). The SADBE solution is administered by wetting the gauze portion of a dermal patch and applying the dermal patch to the inner aspect of the upper arm (the surface facing the chest) and wearing the patch for 2 to 6 hours. Patients are dosed once every three months.

Patients will be eligible for enrollment following any treatment for their cancer, including surgery, chemotherapy, and radiation. Patients with breast cancer, colorectal cancer, gastric cancer, lymphoma, leukemia, glioblastoma, brain cancer, prostate cancer, lung cancer, testicular cancer, renal cell carcinoma, or a sarcoma. The patient population may be selected from one of these cancers or a combination of more than one.

### Results:

Patients receiving the SADBE solution are significantly less likely to have recurrence or progression of their cancer within 12 months study entry than patients receiving placebo.

### Example 4

### Prophetic Example of Prevention of Progression of Prostate Cancer

### Design:

Newly diagnosed patients with prostate cancer treated with only watchful waiting or active surveillance or with ant-androgen therapy are recruited and randomized to receive placebo topical application of SADBE dissolved in dimethylsulfoxide (DMSO) at a concentration of 1% to 2% (weight to volume) or placebo (DMSO alone). The SADBE solution is administered by wetting the gauze portion of a dermal patch and applying the dermal patch to the inner aspect of the upper arm (the surface facing the chest) and wearing the patch for 2 to 6 hours. Patients are dosed once every three months.

### Results:

Patients receiving the SADBE solution are significantly less likely to have progression to require surgery, radiation, chemotheray, or other treatment than those receiving placebo.

Patients receiving the SADBE solution have lower prostate specific antigen (PSA) at 6 months after study entry than those receiving placebo.

Patients receiving the SADBE solution have lower symptoms of prostate cancer than those receiving placebo.

What is claimed is:

1. A method of treating a cancer or precancerous lesions in a mammal comprising:
applying a topical immunosensitizer to skin of the mammal;
wherein the mammal is afflicted with a cancer caused by human papilloma virus (HPV) or precancerous lesions caused by HPV or was afflicted with said cancer and the cancer is now in remission or was afflicted with said precancerous lesions and the lesions have regressed or been removed.

2. The method of claim 1 wherein the method reduces the probability of progression or recurrence of the cancer or progression of the precancerous lesions to cancer.

3. The method of claim 1 wherein the mammal is a human.

4. The method of claim 1 wherein the method does not comprise applying the topical immunosensitizer to a cancerous or precancerous lesion.

5. The method of claim 1 wherein the method comprises applying the topical immunosensitizer to intact skin that is not a site of an HPV lesion.

6. The method of claim 5 wherein the mammal is a human and the administering step comprises applying the topical immunosensitizer to skin on the inner aspect of the upper arm of the human.

7. The method of claim 1 wherein the topical immunosensitizer comprises a squaric acid ester, diphenylcyclopropenone (DPCP), 1-chloro-2,4-dinitrobenzene (DNCB), or 1-chloro-2,6-dinitrobenzene.

8. The method of claim 7 wherein the topical immunosensitizer is squaric acid dibutyl ester (SADBE) and is applied as a solution in a solvent comprising DMSO.

9. The method of claim 1 wherein the human is afflicted with a cancer caused by HPV infection or was afflicted with said cancer and the cancer is now in remission.

10. The method of claim 9 wherein the cancer is in partial remission.

11. The method of claim 9 wherein the cancer is in complete remission.

12. The method of claim 1 wherein the cancer is cervical cancer, head and neck cancer, vulvar cancer, anal cancer, vaginal cancer, or penile cancer and the mammal is afflicted with said cancer or precancerous lesions at risk of progressing to said cancer.

13. A method of treating a mammal for a cancer in remission comprising:
applying a topical immunosensitizer to skin of the mammal;
wherein the mammal has been diagnosed with a cancer, treated for the cancer, and the cancer is in remission at the time of the applying step.

14. A method of treating a mammal for a cancer comprising:
applying a topical immunosensitizer to skin of the mammal.

15. The method of claim 14 wherein the mammal has been diagnosed with a cancer, treated for the cancer, and the cancer is in partial or complete remission at the time of the applying step.

16. The method of claim 15 wherein the cancer is in complete remission at the time of the applying step.

17. The method of claim 14 wherein the mammal is a human.

18. The method of claim 17 wherein the cancer is prostate cancer.

19. The method of claim 17 wherein the cancer is low-grade non-Hodgkins lymphoma.

20. The method of claim 14 wherein the mammal at the time of the applying step is not immunocompromised from any anti-cancer treatments.

21. The method of claim 17 wherein the cancer is breast cancer, colorectal cancer, gastric cancer, lymphoma, leukemia, myelodysplastic syndrome, glioblastoma, brain cancer, prostate cancer, lung cancer, testicular cancer, a sarcoma, lung cancer, renal cell carcinoma, or endometrial cancer.

22. The method of claim 17 wherein the administering step comprises applying the topical immunosensitizer to skin on the inner aspect of the upper arm of the human.

23. The method of claim 14 wherein the topical immunosensitizer comprises a squaric acid ester, diphenylcyclopropenone (DPCP), 1-chloro-2,4-dinitrobenzene (DNCB), or 1-chloro-2,6-dinitrobenzene.

24. The method of claim 23 wherein the topical immunosensitizer is squaric acid dibutyl ester (SADBE) and is applied as a solution in a solvent comprising DMSO.

25. The method of claim 14 wherein the method is a method of preventing recurrence of the cancer;
wherein the mammal was afflicted with said cancer and the cancer is now in remission.

26. The method of claim 14 wherein the method reduces the probability of progression or recurrence of the cancer.
